(19) United States
(12) Patent Application Publication
(10) Pub. No.: US 2024/0341814 A1
(43) Pub. Date: Oct. 17, 2024

(54) ADJUSTABLE DEVICE FOR CORRECTING SCOLIOSIS

(71) Applicant: University Of Cincinnati, Cincinnati, OH (US)

(72) Inventors: William Paris Gaitten, Westerville, OH (US); Olivia Loparo, Rocky River, OH (US); Aaron Burdette, Amelia, OH (US); Viral Jain, Indian Hill, OH (US); Matthew Karp, Loveland, OH (US); Benjamin Wenner, Powell, OH (US); Natalie Yeretzian, Dayton, OH (US); Alexandria Denny, Liberty Township, OH (US); Olivia Rentz, Cincinnati, OH (US); Peter France, Fort Mitchell, KY (US); Richard Casillo, Cincinnati, OH (US)

(21) Appl. No.: 18/635,578

(22) Filed: Apr. 15, 2024

Related U.S. Application Data
(60) Provisional application No. 63/458,953, filed on Apr. 13, 2023.

(51) Int. Cl. A61B 17/70; A61B 17/00; A61B 17/68

(52) U.S. Cl. CPC A61B 17/7014; A61B 2017/00876; A61B 2017/681

(57) ABSTRACT

A distraction device for subcutaneous attachment to vertebrae for correcting curvature of a spine is provided. The distraction device includes a housing that includes a generally hollow interior, a drive system including a drive rod operatively supported in the hollow interior of the housing and a drive element coupled to the drive rod. The drive rod is rotatable within the housing about an axis of rotation and includes a first member a second member. The distraction device includes a first extension arm operatively coupled to the first end of the housing and a second extension arm operatively coupled to the second end of the housing. Rotation of the drive rod, induced by the drive element, causes the first extension arm to move into or out from the opening at the first end of the housing in a first movement direction and the second extension arm to move into or out from the opening at the second end of the housing in a second movement direction to vary a length of the distraction device.

### CROSS-REFERENCE TO RELATED APPLICATION

The present application claims the filing benefit of U.S. Provisional Application Ser. No. 63/458,953, filed Apr. 13, 2023, the disclosure of which is incorporated herein by reference in its entirety.

### TECHNICAL FIELD

The present invention relates generally to surgical implants and, more particularly, to surgical implants for the treatment of scoliosis.

### BACKGROUND

Scoliosis is a general term used to describe the sideways (lateral) curvature of the spine, typically occurring in the thoracic or thoracolumbar region. It is often classified into different treatment categories, including Adolescent Idiopathic Scoliosis, Early Onset Scoliosis, and Adult Scoliosis. Adolescent Idiopathic Scoliosis (AIS) is the most common form of scoliosis, typically diagnosed in adolescents between the ages of 10 and 18 years old, with a higher prevalence in females. It is characterized by a sideways curvature of the spine that develops during the adolescent growth spurt, often without a known cause. AIS can range from mild to severe and may progress during periods of rapid growth.

Chiropractic care, physical therapy, bracing, and surgery are common treatment approaches for AIS. For example, fusion surgery is a common treatment for correcting severe cases of AIS. This procedure involves fusing the vertebrae together with metal rods and screws to straighten the spine. While fusion surgery has been shown to effectively correct the curvature, it comes with drawbacks such as limited spinal flexibility, potential for future complications, large scars, and the need for ongoing medical care.

One of the suspected causes of AIS is thought to be a tight ligamentum flavum, which is a ligament that runs along the length of the spinal column and can contribute to spinal stiffness and curvature. Addressing the tight ligamentum flavum directly through targeted therapies or interventions is a minimally invasive approach to managing AIS. This typically involves techniques to stretch or release the tight ligament, potentially reducing spinal stiffness and curvature.

Given the limitations of current treatment options, there is a need for improved devices and methods for correcting scoliosis, especially AIS, that involve addressing a tight ligamentum flavum.

### SUMMARY

According to an embodiment to the present invention, a distraction device for subcutaneous attachment to vertebrae for correcting curvature of a spine is disclosed. The distraction device includes a housing that having a generally hollow interior. The housing extends longitudinally between an opening to the hollow interior at a first end and an opening to the hollow interior at an opposite second end. The distraction device further includes a drive system including a drive rod operatively supported in the hollow interior of the housing so as to be rotatable therein about an axis of rotation. The drive rod includes a first member at a first end of the drive rod and a second member at an opposite, second end of the drive rod. The drive system further includes a drive element coupled to the drive rod. The drive element is configured to induce rotation of the drive rod about the axis of rotation. The distraction device further includes a first extension arm operatively coupled to the first end of the housing. The first extension arm extends between a first end and an opposite second end and is configured to be movably disposed within the hollow interior of the housing for engagement by the first member of the drive rod. The first extension arm is configured for attachment to a first vertebrae with an anchor assembly. The distraction device further includes a second extension arm operatively coupled to the second end of the housing. The second extension arm extends between a first end and an opposite second end and is configured to be moveably disposed within the hollow interior of the housing for engagement by the second member of the drive rod. The second extension arm is configured for attachment to a second vertebrae with an anchor assembly. Rotation of the drive rod causes the first extension arm to move into or out from the opening at the first end of the housing in a first movement direction and also causes the second extension arm to move into or out from the opening at the second end of the housing in a second movement direction to thereby vary a length of the distraction device.

According to one aspect of the invention, the first extension arm may move linearly in the first movement direction and the second extension arm may move linearly in the second movement direction. Further, the first movement direction may be opposite to the second movement direction.

In one aspect of the invention, the first member of the drive rod includes at least one external thread that extends in a clockwise or counterclockwise direction and the second member of the drive rod includes at least one external thread that extends in an opposite clockwise or counterclockwise direction. Additionally, each second end of the first and the second extension arms may include a threaded socket. The threaded socket of the second end of the first extension arm may be configured to threadably receive the at least one external thread of the first member of the drive rod and the threaded socket of the second end of the second extension arm may be configured to threadably receive the at least one external thread of the second member of the drive rod.

According to another aspect of the invention, the drive system may be configured to receive energy from an external device and the drive element may be configured to receive the conveyed energy to rotate the drive rod in a first rotational direction or an opposite, second rotational direction. For example, the drive element rotates with the drive rod. In one aspect, the drive element may be at least one dipole magnet. For instance, the at least one dipole magnet may be disc-shaped. In yet another aspect, the drive element may include at least two dipole magnets arranged in a stack where the polarities of the at least two dipole magnets are diametrically opposite.

According to one aspect of the invention, the drive rod may include a body arranged axially between the first member and the second member, and the body may be configured to receive the drive element. For example, the body may be a cradle. In another aspect, the housing of the distraction device may include a pair of annular shoulders within the interior of the housing, and the body may be arranged between the pair of annular shoulders to limit axial movement of the drive rod relative to the housing.

In yet another aspect, the second end of the first extension arm may include an annular shoulder that is configured to abut an annular lip at the opening at first end of the housing to retain the second end of the first extension arm within the housing. Additionally, the second end of the second extension arm may include an shoulder configured to abut an annular lip at the opening at second end of the housing to retain the second end of the second extension arm within the housing.

In one aspect of the invention, the first extension arm and the second extension arm may each include a neck between the first end and the second end that is configured to be received by the anchor assembly. For example, the neck may have a smaller cross-sectional area compared to the first and second ends.

In another aspect of the invention, each anchor assembly may be a pedicle screw with a tulip configured to receive the first or second extension arm. In one aspect, the length of the distraction device is configured to span at least one vertebrae to which the distraction device is not attached.

According to another aspect of the invention, a spinal curvature correction system is provided. The spinal curvature correction system includes at least a first distraction device and a second distraction device according to any one of the aspects described above. The spinal curvature correction system further includes a joint member connected at a first end to either the first or second extension arm of the first distraction device and at an opposite second end to either the first or second extension arm of the second distraction device to connect the first and second distraction devices together in series.

In one aspect, the joint member may be configured to bridge over at least one vertebrae to which the distraction device is not attached. In another aspect, the at least one of the first end of the first extension arm or the second extension arm of the first distraction device includes a socket that may be configured to receive the first end of the joint member for connection thereto. Additionally, the at least one of the first end of the first extension arm or the second extension arm of the second distraction device may include a socket configured to receive the second end of the joint member for connection thereto.

According to another aspect, the joint member may be elliptical in transverse cross-sectional shape, including a major axis and a minor axis. Additionally, the major axis of the joint member may be configured to be generally aligned with a frontal plane of a subject and the minor axis may configured to be generally aligned with a sagittal plane of the subject. In that regard, the joint member may provide for up to about 30° of angular deviation between the first distraction device and the second distraction device in the sagittal plane and up to about 10° of angular deviation between the first distraction device and the second distraction device in the frontal plane.

According to another embodiment of the present invention, a method for correcting a curvature in a spine is provided. The method includes providing a distraction device according to any of the aspects described above and subcutaneously implanting the distraction device in a subject by attaching the first extension arm to a first vertebrae with an anchor assembly and attaching the second extension arm to a second vertebrae with an anchor assembly. The method further includes operating the distraction device by applying energy from an external device to the drive element of the drive system to rotate the drive rod in a first rotational direction to lengthen the distraction device or an opposite, second rotational direction to shorten the distraction device, to thereby selectively vary a length of the distraction device to obtain a desired spinal curve.

According to one aspect of the invention, subcutaneously implanting the device further includes implanting the device on a convex portion of the curvature in the spine and operating the distraction device to rotate the drive rod in the first rotational direction to shorten the distraction device. In yet another aspect of the invention, subcutaneously implanting the device includes implanting the device on a concave portion of the curvature in the spine and operating the distraction device to rotate the drive rod in the first rotational direction to lengthen the distraction device.

According to another aspect of the invention, the method further includes providing a second distraction device according to any of the aspects described above and coupling the distraction device and the second distraction device together with a joint member. The joint member may be connected at a first end to either the first or second extension arm of the distraction device and at an opposite second end to either the first or second extension arm of the second distraction device to connect the distraction device and the second distraction device together in series. The method further includes subcutaneously implanting the second distraction device in the subject by attaching the first extension arm to the third vertebrae with an anchor assembly and attaching the second extension arm to the fourth vertebrae with an anchor assembly. The method then includes operating the second distraction device by applying energy from an external device to the drive element of the drive system to rotate the drive rod in a first rotational direction to lengthen the second distraction device or an opposite, second rotational direction shorten the second distraction device, to thereby selectively vary a length of the second distraction device to obtain the desired spinal curve. The distraction device and the second distraction device may be operated independently of one another.

Various additional features and advantages of the invention will become more apparent to those of ordinary skill in the art upon review of the following detailed description of one or more illustrative embodiments taken in conjunction with the accompanying drawings.

### BRIEF DESCRIPTION OF THE DRAWINGS

The accompanying drawings, which are incorporated in and constitute a part of this specification, illustrate one or more embodiments of the invention and, together with the general description given above and the detailed description given below, serve to describe the one or more embodiments of the invention.

FIG. 1 is a diagrammatic posterior view of exemplary subject having a bent scoliotic spine, illustrating the contracting and extending forces applied to the spine with one or more subcutaneous spinal implant systems according to embodiments of the present invention.

FIG. 2 is a posterior view of the subject of FIG. 1, illustrating the subject's straightened spine due to the gradual application of force by selectively extending and contracting the subcutaneous spinal implant system over time according to embodiments of the present invention.

FIG. 3 is a diagrammatic posterior view of a portion of the subject's straightened spine of FIG. 2, illustrating a pair of subcutaneous spinal implant systems mounted on the subject's spine.

FIG. 4 is a schematic perspective view of a pair of distraction devices being connected together to form one of the subcutaneous spinal implant systems of FIG. 3 according to one embodiment of the present invention.

FIG. 5 is a disassembled perspective view of a housing and drive system of a distraction device of FIG. 3.

FIG. 6 is a schematic cross-sectional view of the housing and drive system of FIG. 5, illustrating additional details of a drive rod and a drive element of the drive system.

FIG. 7 is a partial cross-sectional view of a body of the drive rod, illustrating additional details of the drive element.

FIG. 8A is a cross-sectional view of a distraction device of FIG. 3, illustrating the distraction device in a contracted configuration.

FIG. 8B is a view similar to that of FIG. 3, illustrating the distraction device in a extended configuration.

FIG. 9 is a cross-sectional view of two distraction devices connected together in series with a joint member to form a subcutaneous spinal implant system according to one embodiment of the present invention.

FIG. 10 is a cross-sectional view, taken along line 10-10 of FIG. 9, illustrating additional details of the joint member.

FIG. 11 is a schematic perspective view of a distraction devices according to another embodiment of the present invention.

### DETAILED DESCRIPTION

Embodiments of the present invention are directed to a subcutaneous spinal implant system 10, otherwise referred to as a spinal curvature correction system 10, which includes one or more distraction devices 12 connected together in series. The spinal curvature correction system 10 is for treating or otherwise correcting curvature of the spine (i.e., scoliosis, such as AIS) through gradual application of force to a targeted area of the spine. The gradual application of force over time causes slow stretching of biological tissues in the spine, such as the ligamentum flavum, resulting in the straightening of the spine. In that regard, each distraction device 12 is configured to be implantable within the body and mountable to the spine. For example, each distraction device 12 may have two attachment points to vertebrae along the spine, such as to the pedicle of each vertebra, spanning a total of three or more vertebrae. Each distraction device 12 may be attached to the spine using pedicle screw assemblies, and may be attached on either side of the spine. One advantage of the distraction device 12 is the ability to implant it between every other vertebra, for example. This allows for more micromotion within the spine and reduces the number of surgeries needed to implant one or more distraction devices 12. Unlike traditional methods involving vertebral fusion, the present invention allows for surgical spine treatment without fusion, thereby avoiding the drawbacks of vertebral fusion, such as limited spinal flexibility, for example.

Each distraction device 12 is independently and externally controllable to vary a length of the distraction device 12 to thereby apply a force to a part of the subject's spine to which the distraction device 12 is attached. Specifically, each distraction device 12 is actuatable using a magnetically induced drive system within the distraction device 12. The drive system may be rotated using an external device that includes a series of oppositely poled magnets rotating outside of the distraction device 12 and external to the subject. These external magnets induce rotation of one or more magnets of the drive system of the distraction device 12. That is, the magnet(s) within the distraction device 12 are induced into rotation by the larger magnetic system of the external device. The rotational motion of the drive system lengthens (extends) or shortens (contracts) the distraction device 12, as desired. The ability to selectively vary the length of the distraction device 12 allows for the selective application of force to the spine, particularly over time, to thereby achieve a desired spinal curve. To that end, the distraction device 12 of the present invention may be attached to the spine so as to contract on the convex portion of the curve or expand on the concave portion of the spinal curve to approach scoliosis in two ways. Additionally, the distraction device 12, along with the connections between one or more distraction devices 12 connected in series, may be formed of a semi-rigid material to allow for greater degrees of motion in flexion-extension, lateral bending, and axial rotation than would be achievable with a rigid titanium device, for example. These and other benefits of the present invention will be described more fully below.

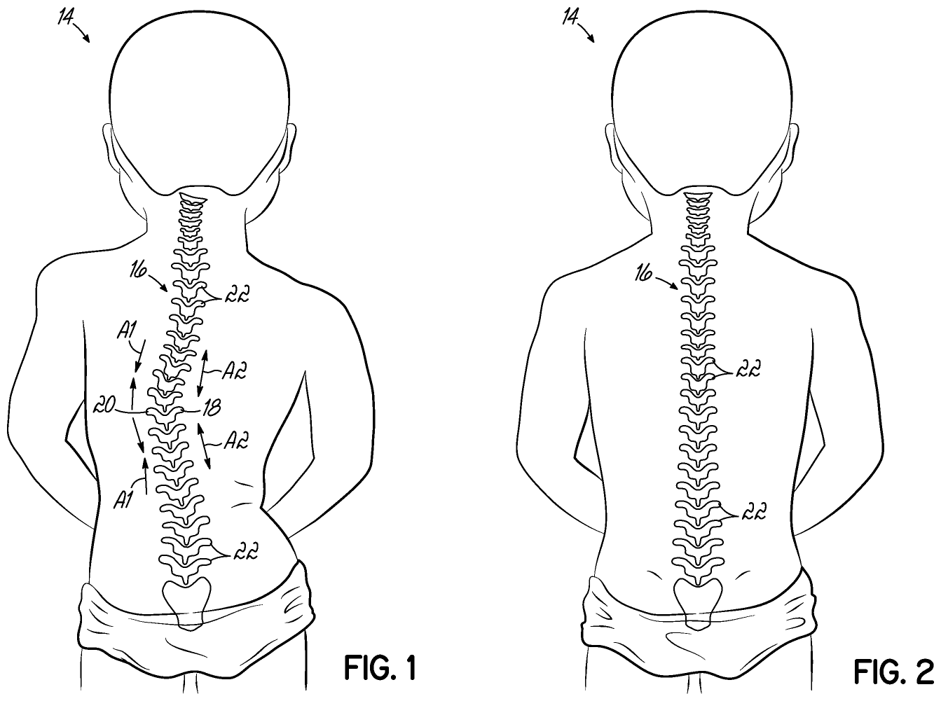

Turning now with reference to the Figures, FIG. 1 is a diagrammatic posterior view of an exemplary subject 14 having a bent scoliotic spine 16. As shown, the subject's 14 spine 16 is laterally curved to define a concave portion 18 of the curvature in the spine 16 and a convex portion 20 of the curvature in the spine 16. In that regard, the distraction device 12 may be attached to vertebrae 22 of the spine 16 so as to be arranged on one side of the spine 16 along the convex portion 20 of the curvature in the spine 16 and/or to vertebrae 22 so as to be arranged on the opposite side of the spine 16 along the concave portion 18 of the curvature in the spine 16. To straighten the subject's 14 scoliotic spine 16, the one or more distraction devices 12 connected along the convex portion 20 of the spine 16 may be operated to shorten or contract the distraction devices 12, as indicated by directional arrows A1. Similarly, the one or more distraction devices 12 connected along the concave portion 18 of the spine 16 may be operated to lengthen or extend the distraction devices 12, as illustrated by directional arrows A2. The gradual application of force to the spine 16, achieved by selectively extending and contracting the distraction devices 12 over time, slowly straightens the subject's 14 spine 16, as shown in FIG. 2.

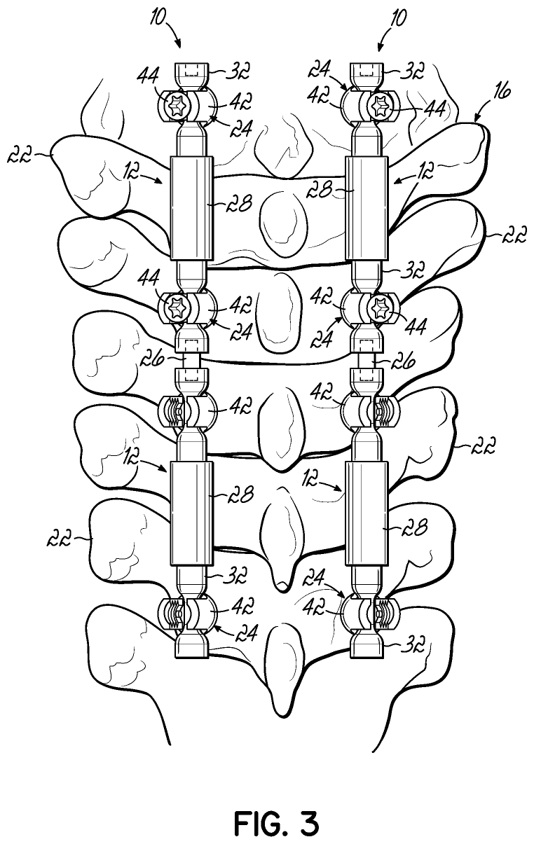

FIG. 3 is an enlarged diagrammatic posterior view of a portion of the subject's 14 spine 16 of FIG. 2. In particular, FIG. 3 shows a pair of subcutaneous spinal implant systems 10 mounted to the subject's 14 spine 16. As shown, one subcutaneous spinal implant system 10 may be attached to vertebrae 22 so as to be arranged on one side of the spine 16 and the other subcutaneous spinal implant system 10 may be attached vertebrae 22 so as to be arranged on the opposite side of the spine 16. In that regard, one subcutaneous spinal implant system 10 may target the convex portion 20 of the curvature in the spine 16 while the other subcutaneous spinal implant system 10 may target the concave portion 18 of the curvature in the spine 16, as described above. Each subcutaneous spinal implant system 10 may be attached to vertebrae 22 using anchor assemblies 24, which may be in the form of pedicle screws, as will be described in further detail below.

While FIG. 3 illustrates a pair of subcutaneous spinal implant systems 10 attached to the subject's 14 spine 16, it will be understood that only one subcutaneous spinal implant system 10 may be attached to the subject's 14 spine 16 to correct the curvature in the spine 16. To that end, the one subcutaneous spinal implant system 10 may be attached to the spine 16 along the convex portion 20 of the spinal curvature or the concave portion 18 of the spinal curvature, and the one or more distraction devices 12 may be operated to either contract or extend, as needed to correct the scoliotic spine 16.

With continued reference to FIG. 3, each subcutaneous spinal implant system 10 includes one or more distraction devices 12 connected in series to form a train of distraction devices 12 along the spine 16 that is the subcutaneous spinal implant system 10. For example, each distraction device 12 may be considered one segment of the subcutaneous spinal implant system 10. The interlinkability of the distraction devices 12 provides the ability to customize the spinal implant system 10 for each subject 14. As shown, the distraction devices 12 of each subcutaneous spinal implant system 10 are connected together with a joint member 26. The joint member 26 may be formed of a flexible semi-rigid material which provides for a certain degree of angular deviation between the two connected distraction devices 12. In that regard, the train of distraction devices 12 may be generally curved to follow the curvature of the scoliotic spine 16. The train of distraction devices 12 may straighten as the distraction devices 12 are operated to straighten the spine 16. While FIG. 3 shows each subcutaneous spinal implant system 10 as having only two distraction devices 12, it will be understood that each subcutaneous spinal implant system 10 may include fewer or more distraction devices 12, as may be needed to correct the curvature in the spine 16.

With continued reference to FIG. 3, each distraction device 12 is attached to two vertebrae 22 to have two fixed points along the spine 16, but spans a total of three or more vertebrae 22. In other words, while each distraction device 12 is anchored to two specific vertebrae 22, the distraction device 12 extends across these two vertebrae 22 as well as intermediate vertebrae 22 in between them. In the exemplary embodiment shown, the distraction device 12 spans three vertebrae 22, however the distraction device 12 may span fewer or more vertebrae 22. Attaching each distraction device 12 to every other or alternating vertebra 22, as shown, allows for more micromotion within the spine 16, thus providing a greater ability to influence the curvature of the spine 16 through operation of the one or more distraction devices 12 of the subcutaneous spinal implant system 10. As shown, each pair of connected distraction devices 12 may span a total of six vertebrae 22. However, by increasing the length of the joint member 26, each pair of connected distraction devices 12 may span a total of seven or more vertebrae 22. For example, the joint member 26 may extend over one or more vertebrae 22 to which the subcutaneous spinal implant system 10 is not attached.

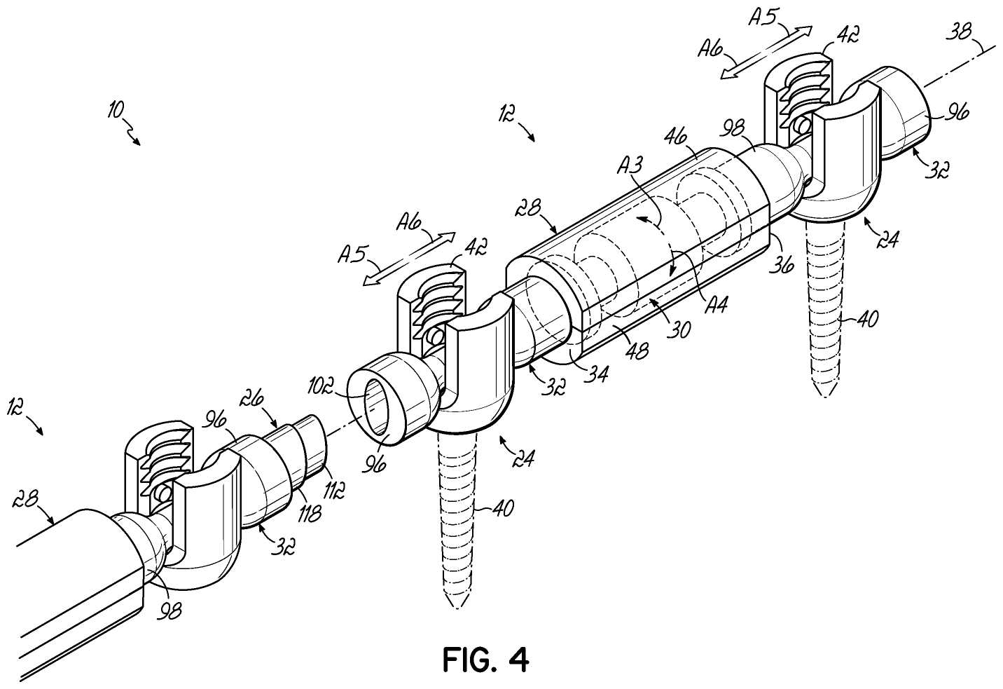

Turning now to FIGS. 4-10, additional details of the subcutaneous spinal implant system 10, and in particular the distraction device 12, will now be described. With reference to FIG. 4, the distraction device 12 includes a housing 28, a drive system 30 housed within the housing 28, and a pair of extension arms 32, each being operatively coupled to an opposite end 34, 36 of the housing 28 so as to be movable into or out from the housing 28 by the drive system 30. Movement of the extension arms 32 in this manner varies a length of the distraction device 12, as will be described in further detail below. The housing 28, drive system 30, and extension arms 32 are arranged coaxially along a longitudinal axis 38 of the distraction device 12. Each component of the distraction device 12 may be made from bioengineered or biological inert materials suitable for resisting biocorrosion once implanted in the subject 14.

Each extension arm 32 of the distraction device 12 is configured to be received by an anchor assembly 24 for securing the distraction device 12 to the spine 16. That is, each extension arm 32 is configured for attachment to a vertebra 22 with an anchor assembly 24. In that regard, the housing 28 is configured to be suspended between the pair of anchor assemblies 24. In the embodiment shown, each anchor assembly 24 includes a screw 40 with a tulip 42 attached to the head of the screw 40. The anchor assembly 24 may be a pedicle screw, for example. The tulip 42 of the anchor assembly 24 includes a slotted body with a threaded socket that is configured to receive a set screw 44 (e.g., FIG. 3). To assemble the anchor assembly 24 to the extension arm 32, the extension arm 32 is inserted into the slotted body of the tulip 42, as shown in FIG. 4, and the set screw 44 is then tightened over the extension arm 32 to establish a secure connection between the extension arm 32 and the tulip 42 of the anchor assembly 24.

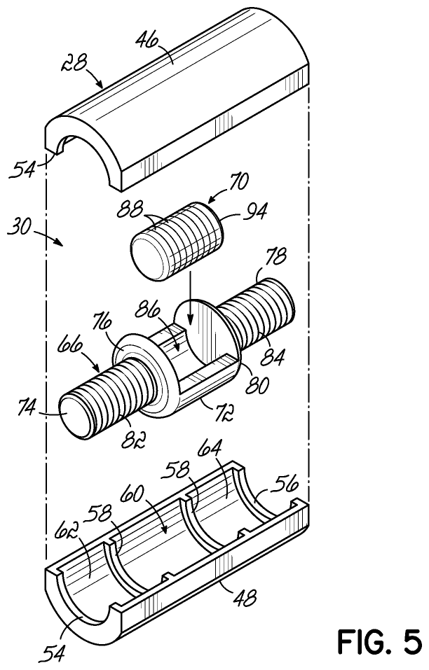

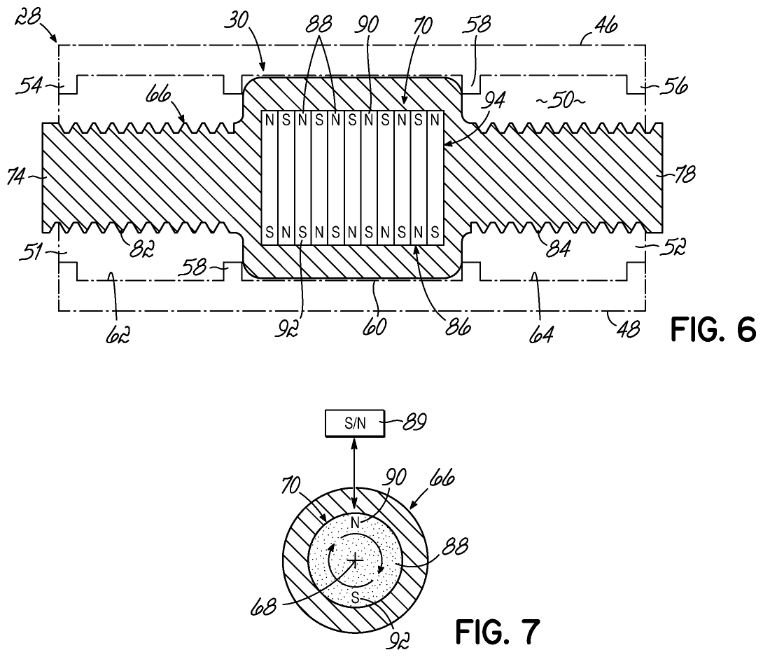

With reference to FIGS. 4-6, the housing 28 extends longitudinally between the first end 34 and the opposite second end 36 to define a length of the housing 28. As shown in FIGS. 5 and 6, the housing 28 includes a first housing half 46 and a second housing half 48 that are configured to be coupled together to define a generally hollowing interior 50 of the housing 28. The housing halves 46, 48 may be coupled together by welding, adhesive bonding, or mechanical fastening such as with suitable fasteners, such as screws or bolts, for example. In an alternative embodiment, the housing 28 may be a single-piece housing, or be formed of more than two parts, for example. Regardless, the housing 28 includes an opening 51 to the hollow interior 50 at the first end 34 of the housing 28 and an opening 52 to the hollow interior 50 at the second end 36 of the housing 28. The opening 51 at the first end 34 of the housing 28 is configured to operatively receive a portion of one extension arm 32 therethrough and the opening 52 at the second end 36 of the housing 28 is configured to operatively receive a portion of the other extension arm 32 therethrough, as will be described in further detail below.

The housing 28 includes a first annular lip 54 that defines the opening 51 to the housing 28 at the first end 34 and a second annular lip 56 that defines the opening 52 to the housing 28 at the second end 36. Within the interior 50, the housing 28 also includes a pair of annular shoulders 58 that are axially spaced apart along the length of the housing 28. In that regard, a drive element chamber 60 is defined between the pair of annular shoulders 58. Furthermore, a first annular guide channel 62 is formed between the first annular lip 54 and one of the annular shoulders 58, the first annular guide channel 62 being adjacent the first end 34 of the housing 28. Likewise, a second annular guide channel 64 is formed between the second annular lip 56 and one the other annular shoulder 58, the second annular guide channel 64 being adjacent the second end 36 of the housing 28. The first and second annular guide channels 62, 64 are configured to slideably receive a portion of each extension arm 32, as will be described in further detail below.

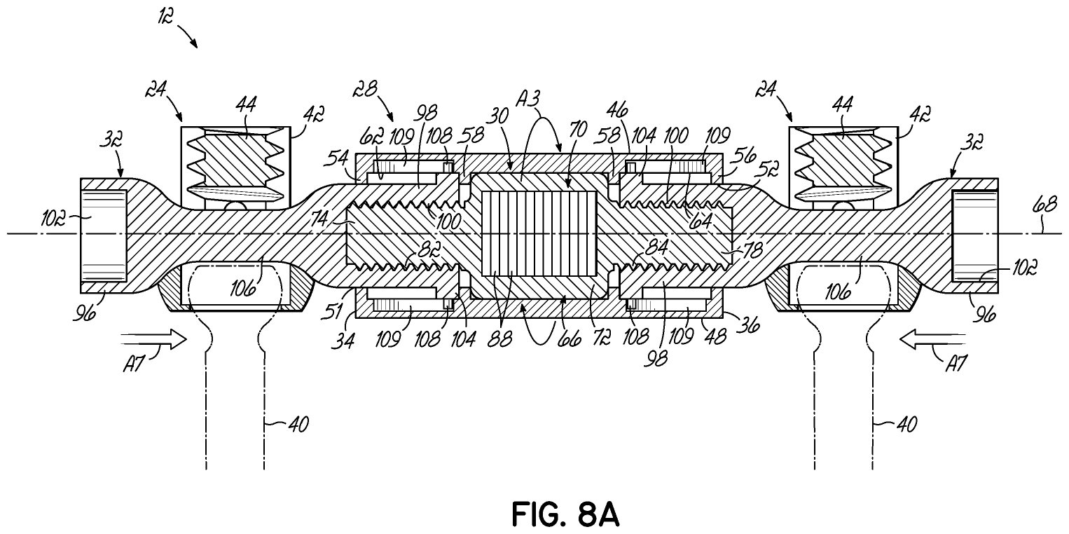

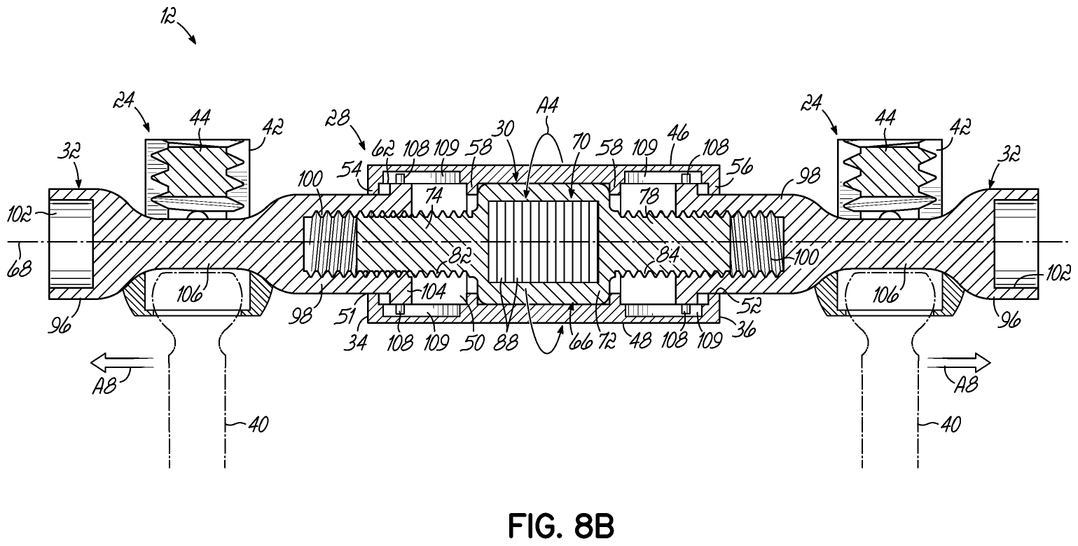

As briefly described above, the distraction device 12 includes the drive system 30 operatively supported within the interior 50 of the housing 28. In particular, the drive system 30 includes a drive rod 66 operatively supported within the interior 50 of the housing 28 for rotation about a rotational axis 68 (e.g., FIGS. 8A and 8B). The drive system 30 further includes a drive element 70 coupled to the drive rod 66 to induce rotation of the drive rod 66 about the rotational axis 68 in both a first rotational direction (e.g., counterclockwise direction) and an opposite second rotational direction (e.g., clockwise direction), as indicated by directional arrows A3 and A4, respectively, in FIG. 4. The drive rod 66 is operatively connected to each extension arm 32 such that rotation of the drive rod 66 in the first rotational direction A3 causes the extension arms 32 to move linearly generally along the longitudinal axis 38 in one direction, as indicated by directional arrow A5 in FIG. 4, and rotation of the drive rod 66 in the second rotational direction A4 causes the extension arms 32 to move linearly generally along the longitudinal axis 38 in the opposite direction, as indicated by directional arrow A6 in FIG. 4.

As best shown in FIG. 5, the drive rod 66 includes a generally cylindrical body 72 with a first externally threaded member 74 projecting from one end 76 of the body and a second externally threaded member 78 projecting from the opposite end 80 of the body 72. In that regard, the body 72 is arranged coaxially between the first and second externally threaded members 74, 78. The body 72 of the drive rod 66 is configured to be received within the drive chamber 60 of the housing 28 (e.g., FIG. 6). In that regard, each end 76, 80 of the body 72 is in a confronting relationship with one of the pair of annular shoulders 58 of the housing 28 to maintain axial alignment of the drive rod 66 within the housing 28. However, the body 72 is able to freely rotate between the pair of annular shoulders 58.

Each externally threaded member 74, 78 of the drive rod 66 is generally cylindrical in shape and extends from the body 72 to a terminal end. In particular, the first and second externally threaded members 74, 78 each include at least one external thread 82, 84, respectively, that extends from the body 72 of the drive rod 66 to the terminal end of the first or second externally threaded member 74, 78. The first and second externally threaded members 74, 78 have opposite threading directions. For instance, the external thread 82 of the first externally threaded member 74 may extend clockwise about the first externally threaded member 74, while the external thread 84 of the second externally threaded member 78 may extend counterclockwise (or vice versa) thereabout. In that regard, rotation of the drive rod 66 in one rotational direction causes each of the first and second externally threaded members 74, 78 to thread into a respective extension arm 32 to draw the extension arms 32 into the housing 28 to thereby shorten the length of the distraction device 12. Rotation of the drive rod 66 in the opposite rotational direction causes each of the first and second externally threaded members 74, 78 to unthread from a respective extension arm 32 to push or move the extension arms 32 out from the housing 28 to lengthen the distraction device 12. Thus, the resultant effect of rotating the drive rod 66 is movement of the extension arms 32 into or out from the housing 28 to vary a length of the distraction device 12.

With continued reference to FIG. 5, the body 72 of the drive rod 66 is configured to support the drive element 70 of the drive system 30. In particular, the body 72 defines a cradle 86 that is configured to receive the drive element 70 such that the drive element 70 is supported by the body 72 within the housing 28. The body 72 is divided longitudinally generally into two halves, including an open top and a rounded bottom that forms the cradle 72. In that regard, the cradle 72 extends a length between ends 76, 80 of the body 72. The cradle 86, and in particular the rounded bottom of the body 72 includes a concave shape to securely hold the drive element 70 within the cradle 86. In an alternative embodiment, the length of the cradle 86 may be longer or shorter, and the cradle 86 may extend more or less circumferentially about the body 72. The cradle 86 may be covered with a cover to enclose the drive element 70 within a chamber formed between the cover and the cradle 86, for example. In either case, the drive element 70 is configured to be attached to the body 72 to transfer rotational motion to the drive rod 66.

The drive element 70 of the drive system 30 is configured to be coupled to the drive rod 66 to induce rotation of the drive rod 66 about its axis of rotation 68. In one embodiment, the drive element 70 may comprise at least one dipole magnet 88. In that regard, an external device 89 (diagrammatically shown in FIG. 7) may be used to externally impart rotational motion to the dipole magnet 88, driving rotation of the drive rod 66 within the housing 28 of the distraction device 12, as briefly described above. The external device 89 may include a motor that rotates two permanent magnets. When the external device 89 is held close to the distraction device 12, energy from the rotating magnets is received by the at least one dipole magnet 88, causing it to rotate. As shown in FIG. 7, the at least one dipole magnet 88 may be a cylinder or disc-shaped dipole magnet. In one embodiment, a single cylinder shaped magnet may be supported in the cradle 86 for attachment to the body 72 of the drive rod 66.

As shown in FIG. 7, the at least one dipole magnet 88 includes two magnetic poles, a north pole 90, and a south pole 92, located on opposite sides of the disc. The magnetic field lines of the dipole magnet 88 run from the north pole 90 to the south pole 92, curving around the edges of the disc. An alternative pole configuration involves axial dipoles, where alternating north and south poles are arranged along the axis of the disc, such as at opposite ends of the disc. To that end, each magnet may have various configurations, including arrangements of alternating north and south poles along the axis (axial multipole) or around the circumference (radial multipole) of the magnet.

As shown, the at least one dipole magnet 88 is arranged within the body 72 of the drive rod 66 so that its rotation is about the rotational axis 68 of the drive rod 66. As a result of the at least one dipole magnet 88 being coupled to the drive rod 66, the energy transferred to the at least one dipole magnet 88 by the external device 89 induces rotation of the drive rod 66 about the rotational axis 68. That is, the at least one dipole magnet 88 is configured to rotate with the drive rod 66. To that end, the drive rod 66 may be selectively rotated in either rotational direction (A2, A3) about the rotational axis 68.

With reference to FIGS. 5 and 6, the drive element 70 in the embodiment shown comprises a stack 94 of dipole magnets 88. The dipole magnets 88 are disc-shaped and are stacked together to form a generally cylindrical-shaped stack 94, which is configured to fit into the cradle 86 for attachment to the body 72 of the drive rod 66. As shown in FIG. 5, the dipole magnets 88 are arranged in the stack 94 such that adjacent dipole magnets 88 in the stack 94 have diametrically opposed polarities. That is, the dipole magnets 88 in the stack 94 are positioned so that the poles of adjacent magnets 88 are diametrically opposed. Arranging the dipole magnets 88 in this way creates a strong magnetic field between each magnet 88. This strong magnetic field between each dipole magnet 88 enhances the interaction between the magnetic fields of the external device and the stack 94 of dipole magnets 88. While the stack 94 of magnets 88 is shown with a specific number of dipole magnets 88 of particular shape and size, it will be understood that the stack 94 may comprise fewer or more dipole magnets 88 of different shapes and sizes.

With reference to FIGS. 4, 8A and 8B, additional details of the extension arms 32 of the distraction device 12 will now be described. In that regard, each extension arm 32 extends a length between a first, operative end 96 and a second, driven end 98 that is configured to be operatively received within the housing 28 of the distraction device 12. The length of each extension arm 32 shown in the Figures is exemplary, and the length of each extension arm 32 may be shorter or longer to vary a total length of the distraction device 12. To that end, the total length of the distraction device 12 may be defined as the distance along the distraction device 12 between the operative ends 96, 98 of the extension arms 32. While the extension arms 32 are depicted with a specific shape and size, it should be understood that other shapes and sizes of the extension arms 32 are also within the scope of the present invention.

As shown, the driven end 98 is movably disposed within the interior 50 of the housing 28 and includes a threaded socket 100 that is configured to threadably receive a respect first or second externally threaded member 74, 78 of the drive rod 66. As a result, each extension arm 32 is operatively connected to the drive system 30 for moving the extension arm 32 relative to the housing 28. The operative end 96 of each extension arm 32 includes a socket 102 that is configured to receive the joint member 26 to connect two distraction devices 12 together, as will be described in further detail below.

The driven end 98 of each extension arm 32 is configured to be movably disposed within the hollow interior 50 of the housing 28, as shown in, for example, FIGS. 8A and 8B. The drive end 98 of each extension arm 32 includes an annular shoulder 104 that is configured to limit movement of the extension arm 32 relative to the housing 28. As shown, the driven end 98 of a first extension arm 32 is inserted through the first opening 51 of the housing 28 and positioned within the first annular channel 62. Specifically, the annular shoulder 104 at the driven end 98 of the extension arm 32 is arranged within the first annular channel 62. The driven end 98 of the extension arm 32 is configured to slide within the first annular channel 62 of the housing 28 from engagement by the drive rod 66. In particular, the extension arm 32 is movable between a fully retracted position where the annular shoulder 104 is in an abutting or near abutting relationship with one of the annular shoulders 58 of the housing 28 and a fully extended position where the annular shoulder 104 of the extension arm 32 is an abutting or near abutting relationship the annular lip 54 at the first end 34 of the housing 28. To that end, the annular lip 54 retains the extension arm 32 within the housing 28. The other extension arm 32 at the second end 36 of the housing 28 is similarly arranged and movable within the second annular channel 64 of the housing 28. The annular shoulder 104 of each extension arm 32 may create a hermetic seal between the housing 28 and the extension arm 32 at each annular channel 62, 64, effectively sealing the interior 50 of the housing 28.

With continued reference to FIGS. 4, 8A and 8B, each extension arm 32 is generally rod-shaped, having a generally cylindrical profile that is circular or roughly elliptical in transverse cross-sectional shape. Each extension arm 32 includes a neck 106 along its length between the driven end 98 and the operative end 96 where a diameter of the extension arm 32 is reduced. In other words, the neck 106 includes a smaller transverse cross-sectional area compared to either the driven end 98 or the operative end 96 of the extension arm 32. As a result, the extension arm 32 is generally “dumbbell” shaped. The neck 106 is configured to be received by the tulip 42 of the anchor assembly 24, as shown in FIG. 4, for example.

To ensure proper orientation of the extension arm 32 within the tulip 42 of the anchor assembly 40, the neck 106 of each extension arm 32 may be shaped or include features such as slotted sides to facilitate correct alignment and positioning of the distraction device 12 relative to the spine 16. The slotted sides may also prevent the extension arms 32 from rotating relative to the tulip 42 of the anchor assembly 24, for example. In one embodiment, only one side of each extension arm 32 may be slotted or flattened, to both prevent rotation of the extension arms 32 relative to the tulip 42, but to also serve as a key or identifier as to the proper orientation of the distraction device 12 for installation in the subject 14. That is, the distraction device 12 may be configured to be installed with the slotted part of each extension arm 32 facing in a specific direction relative to the spine 16, such as facing outwardly from the spine 16, for example. The slotted part of each extension arm 32 indicates to the surgeon implanting the distraction device 12 the proper orientation of the distraction device 12 for each side of the spine 16 (i.e., the convex portion 20 or the concave portion 18 of the curvature of the spine 16). This configuration may be said to provide the distraction device 12 with a “right-handedness” or left-handedness” installation configuration.

Referring now to FIGS. 8A and 8B, operation of one distraction device 12 will now be described. The distraction device 12 may be subcutaneously implanted to a subject's 14 spine 16 by attaching the first extension arm 32 to a first vertebrae 22 with an anchor assembly 24 and attaching a second extension arm 32 to a second vertebrae 22 with an anchor assembly 24, as described above. To that end, the housing 28 may be suspended above a third vertebra 22 between the first and second vertebrae 22. Regardless, the driven end 98 of the first extension arm 32 is movably disposed within the hollow interior 50 of the housing 28 through the opening 51 at the first end 34, with the first externally threaded member 74 of the drive rod 66 threadedly received into the threaded socket 100 of the first extension arm 32. Similarly, the driven end 98 of the second extension arm 32 is movably disposed within the hollow interior 50 of the housing 28 through the opening 52 at the second end 36, with the second externally threaded member 78 of the drive rod 66 threadedly received into the threaded socket 100 of the second extension arm 32.

The distraction device 12 may be operated by applying energy from an external device to the drive element 70 of the drive system 30 to thereby rotate the drive rod 66 in a first rotational direction, as indicated by directional arrows A3 in FIG. 8A. Rotation of the drive rod 66 in the first rotational direction A3 causes the first and second externally threaded members 74, 78 of the drive rod 66 to threadedly engage the threaded socket 100 of a corresponding extension arm 32. As the threaded members 74, 78 are rotated into each socket 100, the extension arms 32 are drawn into the housing 28, in a direction indicated by directional arrows A7, shortening the length of the distraction device 12. The engagement between the driven end 98 of each extension arm 32 and the housing 28 is such that the extension arm 32 may slide linearly relative to the housing 28. The anchor assembly 24 prevents each extension arm 32 from rotation. The distraction device 12 is shown in a fully contracted state in FIG. 8A, where the length of the distraction device 12 is shortest. Operating the distraction device 12 in this manner pulls the pair of anchor assemblies 24 together, manipulating the spinal curvature by affecting the vertebrae 22 to which each anchor assembly 24 is attached. To that end, as each extension arm 32 is drawn toward the body 72 of the drive rod 66, the movement direction of each extension arm 32 is opposite, particularly along the length of the spine 16.

With reference to FIG. 8B, the distraction device 12 may also be operated by applying energy from an external device to the drive element 70 of the drive system 30 to thereby rotate the drive rod 66 in a second rotational direction, as indicated by directional arrows A4; the second rotational direction A4 being opposite of the first rotational direction A3. Rotation of the drive rod 66 in the second rotational direction A4 causes each of the first and second externally threaded members 74, 78 to threadedly disengage (i.e., unthread) from the threaded socket 100 of a corresponding extension arm 32. As the threaded members 74, 78 are rotated out from each socket 100, the extension arms 32 are pushed out from the housing 28, in a direction indicated by directional arrows A8, increasing the length of the distraction device 12. The distraction device 12 is shown in a fully extended state in FIG. 8B, where the length of the distraction device 12 is longest. Operating the distraction device 12 in this manner pushes the pair of anchor assemblies 24 apart, manipulating the spinal curvature by affecting the vertebrae 22 to which each anchor assembly 24 is attached. To that end, as each extension arm 32 is moved out of the housing 28 and away from the body 72 of the drive rod 66, the movement direction of each extension arm 32 is opposite, particularly along the length of the spine 16. The distraction device 12 may be selectively operated to any length between the fully contracted length shown in FIG. 8A and the fully extended length shown in 8B.

The thread directions of the externally threaded members 74, 78 and corresponding sockets 100 may be oppositely threaded compared what is shown and described, and rotation of the drive rod 66 in the first rotational direction A3 may lengthen the distraction device 12 and rotation of the drive rod 66 in the second rotational direction A4 may shorten the distraction device 12. Regardless, rotation of the drive element 70 and thus the drive rod 66 is translated to linear movement of the extension arms 32 as a result of the threaded engagement between the drive rod 66 and the extension arms 32.

As shown in FIGS. 8A and 8B, each extension arm 32 may include a rotation lock feature to prevent rotation of the extension arm 32 relative to the housing 28. In the embodiment shown, the rotation lock feature is in the form of at least one projection 108 that extends radially outwardly from the driven end 98 of the extension arm 32. The projection 108 is configured to slide with a corresponding groove 109 formed in the first and second annular guide channels 62, 64. The groove 109 may extend axially along the housing 28 between the a respective annular shoulder 58 and a respective first or second annular lip 54, 56. In the embodiment shown, each extension arm 32 includes a pair of projections 108 in the form of a pin that projects from the annular shoulder 104 of each extension arm 32. Specifically, the projections 108 are diametrically opposed about the annular shoulder 104 of each extension arm 32. Each projection 108 is configured to be received within a respective groove 109 formed in the annular guide channels 62, 64. To that end, each annular guide channel 62, 64 includes a pair of grooves 109 diametrically opposed about the annular guide channel 62, 64. The projections 108 are permitted to slide axially within each groove 109. However, each groove 109 prevents radial movement of the projections 108 and thus rotational movement of the extension arm 32 relative to the housing 28. Additionally or alternatively, the housing 28 and/or drive rod 66 may include an anti-rotation lock mechanism, such as a magnetically actuated pin, key, locking collar, or other suitable anti-rotation lock mechanism operable to restrict rotation of the drive rod 66 when the drive rod 66 is not receiving energy from the external device.

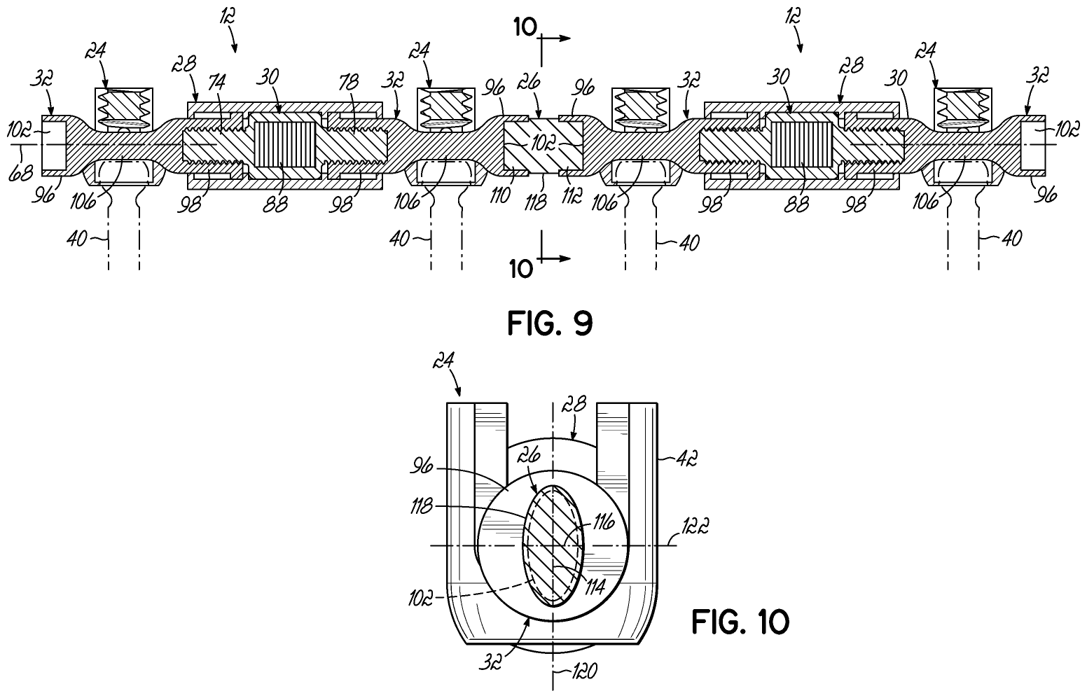

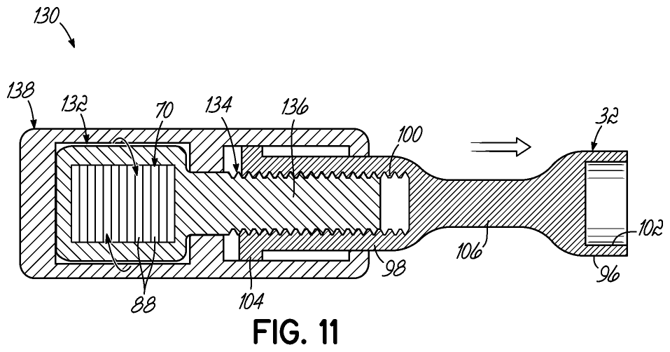

FIG. 9 illustrates a subcutaneous spinal implant system 10 including two distraction devices 12 connected together in series according to one embodiment of the present invention. In that regard, the subcutaneous spinal implant system 10 may have four attachment points to vertebrae 22 along the spine 16. As shown, the distraction devices 12 are connected together with a joint member 26. In particular, the operative end 96 of one extension arm 32 of the first distraction device 12 is connected to the operative end 96 of one extension arm 32 of the second distraction device 12. The joint member 26 extends between a first end 110 and an opposite second end 112 to define a length of the joint member 26. In that regard, the length of the joint member 26 may be sized to bridge over at least one vertebra 22 to which the distraction devices 12 are not attached. Alternatively, the length of the joint member 26 may be sized to bridge between vertebrae 22 to which the distraction devices 12 are attached. In either case, the joint member 26 is shaped as an elliptic cylinder, having a generally elliptical transverse cross-sectional shape, including a major axis 114 and a minor axis 116, as best shown in FIG. 10. The joint member 26 further includes a central band 118 that extends circumferentially about the joint member 26. The central band 118 is an area where a diameter of the joint member 26 is greatest.

As shown in FIG. 9, the first end 110 of the joint member 26 is configured to be fit into the socket 102 of the operative end 96 of the extension arm 32 of the first distraction device 12 and the opposite second end 112 of the joint member 26 is configured to be fit into the socket 102 of the operative end 96 of the extension arm 32 of second first distraction device 12 to connect the first and second distraction devices 12 together in series. Each socket 102 may be generally elliptical in shape to receive a respective end 110, 112 of the joint member 26. The fit between each end 110, 112 of the joint member 26 and the socket 102 of a respective extension arm 32 may be a friction fit to retain the joint member 26 within the socket 102. Additionally or alternatively, the joint member 26 and/or each socket 102 may include a connecting feature, such as a key and keyway, protrusion and groove or slot, or another suitable mechanism, to establish a secure connection therebetween.

The band 118 of the joint member 26 remains external from the socket 102 of each extension arm 32 and is sandwiched between the operative end 96 of each extension arm 32. The band 118 is sized to prevent contact between the operative end 96 of each extension arm 32 to which the joint member 26 is attached. To that end, the joint member 26 may be formed of a flexible semi-rigid material, such as Polyetheretherketone (PEEK), to provide for a certain degree of angular deviation between the two connected distraction devices 12. However, the band 118 is configured to resist flexion or force (i.e., tension and compression) that would cause contact between the connected distraction devices 12 or otherwise result in damage to the distraction devices 12. The size and shape of the band 118 may be varied depending on stress loading conditions expected in each subject 14. The joint member 26 may be formed from one or more other thermoplastic polymers or biocompatible materials suitable to provide for a certain degree of angular deviation between the two connected distraction devices 12.

Referring now to FIG. 10, the joint member 26 is configured to be connected between each distraction device 12 such that when the spinal implant system 10 is attached to the spine 16, the joint member 26 controls flexibility (i.e., angular deviation) between each pair of connected distraction devices 12 in both a frontal plane 120 and a sagittal plane 122 of the subject 14. Specifically, the joint member 26 is arranged to restrict or limit flexibility between connected distraction devices 12 in the frontal plane 120, and permits greater flexibility between connected distraction devices 12 in the sagittal plane 122. In that regard, each distraction device 12 is configured to be attached to the spine 16 such that the major axis 114 of the joint member 26 is generally aligned with the frontal plane 120 of the subject 14, as shown in FIG. 10. The minor axis 116 of the joint member 26 is configured to be generally aligned with the sagittal plane 122 of the subject 14, as shown. As a result, the joint member 26 may provide for up to about 30° of angular deviation between the connected distraction devices 12 in the sagittal plane 122. In comparison, the joint member 26 may provide for up to about 10° of angular deviation between the connected distraction devices 12 in the frontal plane 120. As used herein, the term about means plus or minus 5°.

Referring now to FIG. 11, where like reference numerals represent like features compared to the embodiment of the distraction device 12 described above with respect to FIGS. 1-10, a distraction device 130 is shown according to another embodiment of the present invention. The primary difference between the distraction device 130 of this embodiment and the distraction device 12 of the previous embodiment is that the distraction device 130 only includes a single extension arm 32, rather than a pair of extension arms 32. In that regard, the distraction device 130 includes a drive system 132 with a drive rod 134 and a drive element 70. However, the drive rod 134 only includes a single externally threaded member 136 that is configured to threadedly engage the threaded socket 102 of the extension arm 32 to extend or retract the extension arm 32 from a housing 138. In that regard, and like the embodiment of the distraction device 12 described above, an external device may be operated to induce rotation of the drive rod 134 in a first rotational direction to draw the extension arm 32 into the housing 138 to shorten the distraction device 130. Rotation of the drive rod 134 in the opposite rotational direction moves the extension arm 32 out from the housing 138 to lengthen the distraction device 130.

The distraction device 130 may be attachable to the spine 16 with a pair of anchor assemblies 24. In particular, the extension arm 32 may be attached to a first vertebra 22 with one anchor assembly 24 and the housing 138 of the distraction device 130 may be attached to a second, adjacent vertebra 22 with the other anchor assembly 24. In that regard, the distraction device 130 is configured to be placed intervertebrally. Additionally, the distraction device 130 may be connected in series to other distraction devices 130, with a joint member 26, for example, to form a train of distraction devices 130 along the spine 16 referred to as a subcutaneous spinal implant system.

While the invention has been illustrated by the description of various embodiments thereof, and while the embodiments have been described in considerable detail, it is not intended to restrict or in any way limit the scope of the appended claims to such detail. Thus, the various features discussed herein may be used alone or in any combination. Additional advantages and modifications will readily appear to those skilled in the art. The invention in its broader aspects is therefore not limited to the specific details and illustrative examples shown and described. Accordingly, departures may be made from such details without departing from the scope of the general inventive concept.

What is claimed is:

1. A distraction device for subcutaneous attachment to vertebrae for correcting curvature of a spine, comprising:
a housing that includes a generally hollow interior, the housing extending longitudinally between an opening to the hollow interior at a first end and an opening to the hollow interior at an opposite second end;
a drive system, comprising:
a drive rod operatively supported in the hollow interior of the housing so as to be rotatable therein about an axis of rotation, the drive rod including a first member at a first end of the drive rod and a second member at an opposite, second end of the drive rod; and
a drive element coupled to the drive rod, the drive element being configured to induce rotation of the drive rod about the axis of rotation; and
a first extension arm operatively coupled to the first end of the housing, the first extension arm extending between a first end and an opposite second end configured to be movably disposed within the hollow interior of the housing for engagement by the first member of the drive rod, the first extension arm being configured for attachment to a first vertebrae with an anchor assembly; and
a second extension arm operatively coupled to the second end of the housing, the second extension arm extending between a first end and an opposite second end configured to be moveably disposed within the hollow interior of the housing for engagement by the second member of the drive rod, the second extension arm being configured for attachment to a second vertebrae with an anchor assembly;
wherein rotation of the drive rod causes the first extension arm to move into or out from the opening at the first end of the housing in a first movement direction and the second extension arm to move into or out from the opening at the second end of the housing in a second movement direction to vary a length of the distraction device.

2. The distraction device of claim 1, wherein the first extension arm moves linearly in the first movement direction, and the second extension arm moves linearly in the second movement direction.

3. The distraction device of claim 1, wherein the first movement direction is opposite to the second movement direction.

4. The distraction device of claim 1, wherein the first member of the drive rod includes at least one external thread that extends in a clockwise or counterclockwise direction and the second member of the drive rod includes at least one external thread that extends in an opposite clockwise or counterclockwise direction.

5. The distraction device of claim 4, wherein each second end of the first and the second extension arms includes a threaded socket, the threaded socket of the second end of the first extension arm being configured to threadably receive the at least one external thread of the first member of the drive rod and the threaded socket of the second end of the second extension arm being configured to threadably receive the at least one external thread of the second member of the drive rod.

6. The distraction device of claim 1, wherein the drive system is configured to receive energy from an external device, wherein the drive element is configured to receive the conveyed energy to rotate the drive rod in a first rotational direction or an opposite, second rotational direction.

7. The distraction device of claim 6, wherein the drive element rotates with the drive rod.

8. The distraction device of claim 1, wherein the drive element comprises at least one dipole magnet.

9. The distraction device of claim 8, wherein the at least one dipole magnet is disc-shaped.

10. The distraction device of claim 1, wherein the drive element comprises at least two dipole magnets arranged in a stack where the polarities of the at least two dipole magnets are diametrically opposite.

11. The distraction device of claim 1, wherein the drive rod includes a body arranged axially between the first member and the second member, the body being configured to receive the drive element.

12. The distraction device of claim 11, wherein the body comprises a cradle.

13. The distraction device of claim 11, wherein the housing includes a pair of annular shoulders within the interior of the housing, the body being arranged between the pair of annular shoulders to limit axial movement of the drive rod relative to the housing.

14. The distraction device of claim 1, wherein the second end of the first extension arm includes an annular shoulder configured to abut an annular lip at the opening at first end of the housing to retain the second end of the first extension arm within the housing, and wherein the second end of the second extension arm includes an annular shoulder configured to abut an annular lip at the opening at second end of the housing to retain the second end of the second extension arm within the housing.

15. The distraction device of claim 1, wherein the first extension arm and the second extension arm each include a neck between the first end and the second end that is configured to be received by the anchor assembly, the neck having a smaller cross-sectional area compared to the first and second ends.

16. The distraction device of claim 1, wherein each anchor assembly comprises a pedicle screw with a tulip configured to receive the first or second extension arm.

17. The distraction device of claim 1, wherein the length of the distraction device is configured to span at least one vertebrae to which the distraction device is not attached.

18. A spinal curvature correction system, comprising:
at least a first distraction device and a second distraction device according to claim 1; and
a joint member connected at a first end to either the first or second extension arm of the first distraction device and at an opposite second end to either the first or second extension arm of the second distraction device to connect the first and second distraction devices together in series.

19. The spinal curvature correction system of claim 18, wherein the joint member is configured to bridge over at least one vertebrae to which the distraction device is not attached.

20. The spinal curvature correction system of claim 18, wherein at least one of the first end of the first extension arm or the second extension arm of the first distraction device includes a socket configured to receive the first end of the joint member for connection thereto, and wherein at least one of the first end of the first extension arm or the second extension arm of the second distraction device includes a socket configured to receive the second end of the joint member for connection thereto.

21. The spinal curvature correction system of claim 18, wherein the joint member is elliptical in transverse cross-sectional shape, including a major axis and a minor axis.

22. The spinal curvature correction system of claim 21, wherein the major axis of the joint member is configured to be generally aligned with a frontal plane of a subject and the minor axis is configured to be generally aligned with a sagittal plane of the subject.

23. The spinal curvature correction system of claim 22, wherein the joint member provides for up to about 30° of angular deviation between the first distraction device and the second distraction device in the sagittal plane and up to about 10° of angular deviation between the first distraction device and the second distraction device in the frontal plane.

24. A method for correcting a curvature in a spine, comprising:
providing a distraction device, comprising:
a housing that includes a generally hollow interior, the housing extending longitudinally between an opening to the hollow interior at a first end and an opening to the hollow interior at an opposite second end;
a drive system, comprising:
a drive rod operatively supported in the hollow interior of the housing so as to be rotatable therein about an axis of rotation, the drive rod including a first member at a first end of the drive rod and a second member at an opposite, second end of the drive rod; and
a drive element coupled to the drive rod, the drive element being configured to induce rotation of the drive rod about the axis of rotation; and
a first extension arm operatively coupled to the first end of the housing, the first extension arm extending between a first end and an opposite second end configured to be movably disposed within the hollow interior of the housing for engagement by the first member of the drive rod, the first extension arm being configured for attachment to a first vertebrae with an anchor assembly; and
a second extension arm operatively coupled to the second end of the housing, the second extension arm extending between a first end and an opposite second end configured to be moveably disposed within the hollow interior of the housing for engagement by the second member of the drive rod, the second extension arm being configured for attachment to a second vertebrae with an anchor assembly;
subcutaneously implanting the distraction device by attaching the first extension arm to a first vertebrae with an anchor assembly and attaching the second extension arm to a second vertebrae with an anchor assembly; and
operating the distraction device by applying energy from an external device to the drive element of the drive system to rotate the drive rod in a first rotational direction to lengthen the distraction device or an opposite, second rotational direction to shorten the distraction device, to thereby selectively vary a length of the distraction device to obtain a desired spinal curve.

25. The method of claim 24, wherein subcutaneously implanting the device further comprises implanting the device on a convex portion of the curvature in the spine, and operating the distraction device to rotate the drive rod in the first rotational direction to shorten the distraction device.

26. The method of claim 24, wherein subcutaneously implanting the device further comprises implanting the device on a concave portion of the curvature in the spine, and operating the distraction device to rotate the drive rod in the first rotational direction to lengthen the distraction device.

27. The method of claim 24, further comprising:
providing a second distraction device, comprising:
a housing that includes a generally hollow interior, the housing extending longitudinally between an opening the hollow interior at a first end and an opening to the hollow interior at an opposite second end;
a drive system, comprising:
a drive rod operatively supported in the hollow interior of the housing so as to be rotatable therein about an axis of rotation, the drive rod including a first member at a first end of the drive rod and a second member at an opposite, second end of the drive rod; and
a drive element coupled to the drive rod to drive rotation of the drive rod about the axis of rotation; and
a first extension arm operatively coupled to the first end of the housing, the first extension arm extending between a first end and an opposite second end configured to be movably disposed within the hollow interior of the housing for engagement by the first member of the drive rod, the first extension arm being configured for attachment to a third vertebrae with an anchor assembly; and
a second extension arm operatively coupled to the second end of the housing, the second extension arm extending between a first end and an opposite second end configured to be moveably disposed within the hollow interior of the housing for engagement by the second member of the drive rod, the second extension arm being configured for attachment to a fourth vertebrae with an anchor assembly;
coupling the distraction device and the second distraction device together with a joint member, the joint member being connected at a first end to either the first or second extension arm of the distraction device and at an opposite second end to either the first or second extension arm of the second distraction device to connect the distraction device and the second distraction device together in series;
subcutaneously implanting the second distraction device by attaching the first extension arm to the third vertebrae with an anchor assembly and attaching the second extension arm to the fourth vertebrae with an anchor assembly; and
operating the second distraction device by applying energy from an external device to the drive element of the drive system to rotate the drive rod in a first rotational direction to lengthen the second distraction device or an opposite, second rotational direction shorten the second distraction device, to thereby selectively vary a length of the second distraction device to obtain the desired spinal curve.

28. The method of claim 27, wherein the distraction device and the second distraction device are operated independently of one another.
